(19) United States
(12) Patent Application Publication
(10) Pub. No.: US 2009/0209617 A1
(43) Pub. Date: Aug. 20, 2009

(54) DULOXETINE SALTS

(76) Inventors: Tibor Mezei, Budapest (HU); Gyula Simig, Budapest (HU); Eniko Molnar, Erd (HU); Miklos Szabo, Godollo (HU); Gyula Lukacs, Budapest (HU); Marta Porcs-Makkay, Pomaz (HU); Erika Szilagyi, Tura (HU); Tibor Bako, Budapest (HU)

Correspondence Address: K.F. ROSS P.C., 5683 RIVERDALE AVENUE, SUITE 203 BOX 900, BRONX, NY, 10471-0900, US

(21) Appl. No.: 12/282,914

(22) PCT Filed: Mar. 13, 2007
(86) PCT No.: PCT/HU07/00025

§ 371 Date: Apr. 28, 2009

(30) Foreign Application Priority Data
Mar. 13, 2006 (HU) P0600198

(51) Int. Cl. A61K 31/381; C07D 333/20; G01N 30/02; C07D 333/16; A61P 25/24; A61P 25/22

(52) U.S. Cl. 514/438; 549/75; 436/161

(57) ABSTRACT

The subject of the present invention is the provision of new salts of duloxetine of the Formula (I) with organic acids, process for their preparation and medicinal products containing thereof. The new salts are essentially free from the impurity of the Formula (II) and possess high purity and high stability. The new duloxetine salts are prepared by reacting duloxetine free base dissolved in an organic solvent with an approximately equimolar amount of an organic acid. Particularly advantageous crystalline salts are those formed with fumaric acid, citric acid or (−)-mandelic acid.

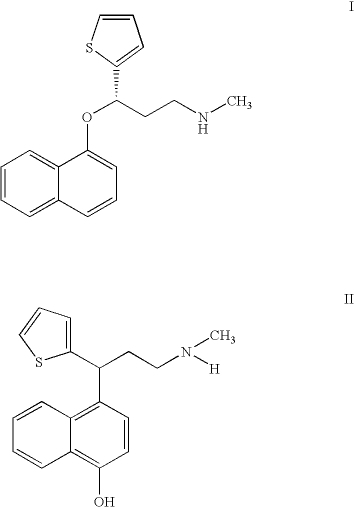

### FIELD OF THE INVENTION

The present invention relates to new, high purity salts of (+)-(S)—N-methyl-3-(1-naphtyloxy)-3-(2-thienyl)-propylamine of the Formula (I),

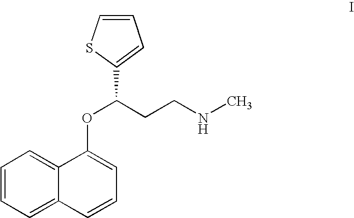

preparation thereof and their use as active pharmaceutical ingredients.

### TECHNICAL BACKGROUND OF THE INVENTION

(+)-(S)—N-methyl-3-(1-naphtyloxy)-3-(2-thienyl)-propylamine of the Formula (I) is known under the International Nonproprietary Name duloxetine. Duloxetine is a selective inhibitor of serotonine and norepinephrine uptake in the human body, and it is used in the clinical practice as antidepressant.

One of the main causes of depression and anxiety is the decreased concentration of serotonine in the central nervous system. The serotonine concentration can either be influenced by the enhancement of the serotonine biosynthesis or by inhibiting the metabolism of serotonine. Duloxetine is an efficient active pharmaceutical ingredient inhibiting the decomposition of serotonine. Duloxetine of the Formula (I) belongs to the group of selective serotonine-norepinephrine reuptake inhibitors (SSNRIs).

Chemically, duloxetine is an optically active compound. Its antipode has also been prepared (Tetrahedron Lett. 1990, 31, 7101-04).

Several acid addition salts of duloxetine are known from the state of the art. During the evaluation of the pharmacological activity, the oxalate salt of duloxetine was used. However, it has been established later that the hydrochloride salt of duloxetine prepared according to the state of the art is more suitable for the use in the therapy (Drugs of the Future 2000. 25 (9) 907-916). Furthermore, according to the state of the art, the maleate salt has also been disclosed. (European Patent No. 273658).

Published International Patent Application No. WO 2004/056795 discloses a process for the resolution of racemic duloxetine of the Formula (I) using (−)-ditoluyl-tartaric acid. The inventors provided a process for the racemization and recycling of the non-desired enantiomer, which increased the economy of the process. However, the physical properties of the salt of duloxetine with (−)-ditoluyl-tartaric acid have not been disclosed.

Marketed medicinal products have to comply with several strict criteria specified in pharmacopoeias. It is the duty of the producer to prove the compliance with the regulations set forth before the health authority prior to the issue of the marketing authorization. Some regulations are related to the active pharmaceutical ingredient, while others apply to the medicinal product itself. During the development process, both sets of criteria are tightly interconnected taking into account that the final aim is the launch and subsequent marketing of a new medicinal product.

Several criteria set forth by the health authorities with regard the active pharmaceutical ingredient used in the medicinal product is related with chemical purity. In most cases, said active ingredients are high molecular weight organic bases, which are poorly soluble in or wettable with water. The hydrophobicity of the substance can be the source of problems during formulation, especially in preparing tablets. Therefore, most active pharmaceutical ingredients are prepared and used in the form of salts.

Transformation of the base of the active ingredient into salt is advantageous in two respects.

In general, salts have higher melting temperatures than the corresponding free bases, therefore salts can be purified more efficiently and conveniently. The convenience of the purification process is justified by the strict requirements towards purity of the active pharmaceutical ingredient.

The second advantage of the use of a salt form resides in the fact that in general, salts are more soluble in and are much more wettable with water than the corresponding free bases. This fact is advantageous during the development and production of the medicinal product.

One of the most important criteria set forth for the marketed medicinal product is stability. Stability means that neither the decrease in the content of the active ingredient, nor the increase in the concentration of the impurities and by-products present in the medicinal product should exceed the limit value during the manufacture or the shelf-life of the product. The assurance of the stability of a medicinal product is a complicated task, since the conditions applied during manufacture may result in the decomposition of the active ingredient. Furthermore, in the presence and under the influence of the pharmaceutical carriers and other auxiliary agents present in the finished dosage form, various decomposition processes can take place. Such decomposition processes can be especially severe in the case when the active pharmaceutical ingredient is present in micronized or highly dispersed form.

During the process of stability testing, the identity and concentration of individual impurities present in the medicinal product are determined as the function of time and storage conditions. In the aforementioned tests, generally high-performance liquid chromatography and mass spectrometry are used as analytical methods.

During our investigations regarding duloxetine, we have found that during the stability testing of tablets containing duloxetine hydrochloride, several impurities can be identified. In order to identify the above mentioned impurities, each of them has been separated by high-performance liquid chromatography and their molecular weight was determined by mass spectrometry. Surprisingly, we have found that the molecular weight of the most significant impurity is identical to that of duloxetine of the Formula (I), which indicates that the impurity is produced in a rearrangement process.

After the review of the prior art, we concluded that at high temperature or under the effect of a strong mineral acid, phenyl- or naphtyl-ethers can undergo the so-called Claisen-rearrangement, which results in the formation of a product having the same molecular weight as the starting compound. High-temperature Claisen-rearrangement of naphtyl-ethers is a known process according to the state of the art (Behaghel; Chem. Berichte 67; 1934; 1368). It is also concluded from the prior art that the Claisen-rearrangement is catalyzed by a strong mineral acid, thus the rearrangement may proceed even at lower temperature (Lutz P.; Chem. Rew. 1984. Vol. 84. No. 3, 205-247).

On the basis of the above mentioned facts, we presumed that the impurity observed during the stability testing of duloxetine hydrochloride-containing tablets having identical molecular weight to that of duloxetine corresponds to the compound of the Formula (II),

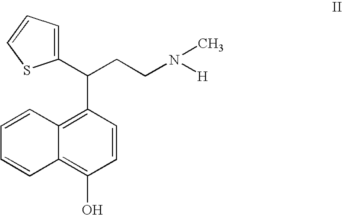

which is formed by the rearrangement of duloxetine in presence of a strong acid.

During further investigations, we have determined that duloxetine is especially sensitive to certain acidic conditions. Very surprisingly, we have found that during the preparation of salts of duloxetine with strong mineral acids, especially with hydrobromic acid, the formation of the compound of the Formula (II) occurs in a few minutes at the temperature of 25 to 50° C., which is surprisingly low temperature as compared to the temperature of Claisen-rearrangement known from the prior art.

Subsequently, the compound of the Formula (II) has been prepared by the rearrangement of duloxetine hydrobromide in pure form and in good yield and the thus obtained product was subjected to structure elucidation. The identity of the thus obtained (±)-N-methyl-3-(2-thienyl)-3-(4-hydroxy-1-naphtyl)-propylamine of the Formula (II) with the impurity observed during the stability testing has been established using high-performance liquid chromatography and mass spectrometry.

On the basis of the above facts, we expected that (±)-N-methyl-3-(2-thienyl)-3-(4-hydroxy-1-naphtyl)-propylamine of the Formula (II) is produced during the shelf-life of a duloxetine hydrochloride-containing medicinal product and that said compound of the Formula (II) may be the most significant impurity and critical factor of the stability of the product.

As a result of the above investigations, we have determined that it is desirable to prepare high-purity duloxetine suitable for the preparation of medicinal products in a form that is exempt from the presence of strong mineral acids, for example, wherein a salt form of duloxetine is different from its hydrochloride or hydrobromide salt.

### SUMMARY OF THE INVENTION

The technical problem to be solved by the present invention was to provide new, pharmaceutically acceptable salts of duloxetine of the Formula (I), which are devoid from (±)-N-methyl-3-(2-thienyl)-3-(4-hydroxy-1-naphtyl)-propylamine of the Formula (II) and which are suitable by physical and biological properties for the preparation of medicinal products.

The above objective is solved by the present invention.

During our investigations, it has been surprisingly found that salts of (+)-(S)—N-methyl-3-(1-naphtyloxy)-3-(2-thienyl)-propylamine of the Formula (I) with organic acids are especially suitable for the production of medicinal product and that the above-mentioned salts can be prepared in such a way that the formation of (±)-N-methyl-3-(2-thienyl)-3-(4-hydroxy-1-naphtyl)-propylamine of the Formula (II) is prevented.

### DETAILED DESCRIPTION OF THE INVENTION

In the field of pharmaceutical chemistry, it is common practice to use organic acids for salt formation of biologically active organic bases instead of a strong inorganic acid.

According to the first aspect of the present invention, there are provided salts of duloxetine of the Formula (I) with organic acid, wherein the formation of (±)-N-methyl-3-(2-thienyl)-3-(4-hydroxy-1-naphtyl)-propylamine of the Formula (II) is inhibited, thus being essentially free from the impurity of the Formula (II). The limit of detection for the compound of the Formula (II) is a feature of the high-performance liquid chromatographic method used for the assay thereof. Using the chromatographic method disclosed in the present application, said limit of detection is 0.01 percent by weight. The new salts of duloxetine possess more favourable stability characteristics than duloxetine salts formed with inorganic acids known from the prior art.

The organic acid used for salt formation is selected from formic acid, acetic acid, propionic acid, maleic acid, fumaric acid, succinic acid, lactic acid, malic acid, tartaric acid, citric acid, ascorbic acid, malonic acid, oxalic acid, mandelic acid, glycolic acid, phtalic acid, benzenesulphonic acid, toluenesulphonic acid, naphtalenesulphonic acid, or methanesulphonic acid. Preferably, the organic acid is fumaric acid, citric acid or mandelic acid.

Particularly preferably salts of duloxetine of the Formula (I) with organic acids according to the present invention are the salts with fumaric acid, citric acid and (−)-mandelic acid. Said salts are free from the impurity (±)-N-methyl-3-(2-thienyl)-3-(4-hydroxy-1-naphtyl)-propylamine of the Formula (II). Said salts are particularly suitable for the preparation of medicinal products. Formation of the compound (±)-N-methyl-3-(2-thienyl)-3-(4-hydroxy-1-naphtyl)-propylamine of the Formula (II) can be detected neither during the manufacture of the above-mentioned particularly preferable salts, nor during the shelf-life of the medicinal products prepared therefrom. Fumaric acid, citric acid and (−)-mandelic acid belong to the group of the weak organic acids, which are significantly weaker then the inorganic mineral acids and oxalic acid as well.

According to a further aspect of the present invention, there is provided a process for the preparation of fumarate, citrate or (−)-mandelate salts of duloxetine of the Formula (I), which comprises mixing the solution of duloxetine free base of the Formula (I) prepared in an organic solvent with an approximately equimolar amount of the organic acid, and the mixture is heated until the acid is completely dissolved. Complete dissolution is generally achieved when the mixture is heated until its boiling temperature. Once the organic acid has dissolved, the solution is cooled, the salt of duloxetine of the Formula (I) according to the present invention is precipitated upon cooling and isolated by filtration.

In the process for the preparation of duloxetine fumarate and citrate, less polar organic solvents, for example, ethylacetate, diethylether or acetone, preferably acetone can be used. The organic acid component is used in 1.0-1.2 molar-equivalent, preferably 1.0 molar-equivalent amount related to the amount of duloxetine of the Formula (I).

The important advantage of the salt of duloxetine with (−)-mandelic acid resides in the fact that said salt is suitable for the resolution of racemic duloxetine, or in the case when the optical purity of the crude duloxetine base used for salt formation is not satisfactory, the preparation of the salt of duloxetine with (−)-mandelic acid is an effective method for achieving significant increase in optical purity.

The preparation of duloxetine (−)-mandelate according to the present invention can be carried out in organic solvents, such as alcohols, acetone or ethylacetate. As alcohol, an aliphatic alcohol comprising 1 to 4 carbon atoms, e.g. methanol, ethanol, 2-propanol can be used. Preferably, the preparation of duloxetine (−) mandelate is carried out in water-free ethanol. In the case when optically pure duloxetine base is used as starting component, (−)-mandelic acid is used in 1.0-1.1 molar equivalent amount related to the amount of duloxetine of the Formula (I).

According to the present invention, there is provided a process for the resolution of racemic duloxetine of the Formula (I) with (−)-mandelic acid. In this case, (−)-mandelic acid is used in 0.5-1.0 molar-equivalent, preferably, in 0.60-0.75 molar equivalent amount relative to the amount of duloxetine base. The yield of the primary product duloxetine (−)-mandelate increases with the amount of the acid used. The optical purity of the crude product decreases with increasing yield. The optimal yield and the desired optical purity is achieved when 0.60-0.75 molar equivalents of (−)-mandelic acid are used.

A further subject of the present invention is (±)-N-methyl-3-(2-thienyl)-3-(4-hydroxy-1-naphtyl)-propylamine of the Formula (II), its isotope-labelled analogs, wherein the labeling isotope is selected from deuterium, tritium, ¹³C, ¹⁴C, ¹⁵N, ¹⁷O, ¹⁸O, ³³S, ³⁴S or ³⁶S, and acid addition salts thereof. The analog isotope-labelled compounds corresponding to the Formula (I) can be prepared according to processes known from the prior art. (±)-N-methyl-3-(2-thienyl)-3-(4-hydroxy-1-naphtyl)-propylamine of the Formula (II) or isotope-labelled analogs and salts thereof are chemical reference substances suitable in the chemical testing of duloxetine active pharmaceutical ingredient and medicinal products containing thereof.

According to a further aspect of the present invention, there is provided a process for the preparation of (±)-N-methyl-3-(2-thienyl)-3-(4-hydroxy-1-naphtyl)-propylamine of the Formula (II). Preparation of (±)-N-methyl-3-(2-thienyl)-3-(4-hydroxy-1-naphtyl)-propylamine of the Formula (II) is carried out in a polar solvent, e.g. in an alcohol comprising 1 to 4 carbon atoms, i.e. methanol, ethanol, 2-propanol, 1-butanol; acetic acid, water or mixture thereof in the presence of a strong mineral acid.

Any strong mineral acid, e.g. hydrochloric acid, sulphuric acid, hydrogen bromide can be used in the reaction. Preferably hydrogen bromide is used. The amount of the mineral acid can be chosen between 1 to 5 molar-equivalents relative to the amount of the starting substance. Preferably, 2 molar equivalents of the mineral acid are used.

In the case when hydrogen bromide is applied, it can be provided in the gaseous state or in the form of an aqeuous or acetic acid solution thereof.

The reaction time for the rearrangement reaction is a few hours at the temperature of 25° C. At higher temperature, the reaction may be completed in a few minutes.

According to a further aspect of the present invention, there is provided a method for the assay of (±)-N-methyl-3-(2-thienyl)-3-(4-hydroxy-1-naphtyl)-propylamine of the Formula (II) in duloxetine active pharmaceutical ingredient and finished medicinal product containing thereof. The assay of the compound of the Formula (II), which is a potential impurity of duloxetine, is performed by high-performance liquid chromatography.

In the assay of the compound of the Formula (II), (±)-N-methyl-3-(2-thienyl)-3-(4-hydroxy-1-naphtyl)-propylamine prepared according to the present invention is used in the pure form as chemical reference substance.

The quantitative assay is generally performed by external calibration method. However, other calibration methods, such as internal standard method or standard addition method, which are all known from the prior art, can be used.

As a detection method, any method capable to provide an analytical signal suitable for the detection of duloxetine of the Formula (I) and (±)-N-methyl-3-(2-thienyl)-3-(4-hydroxy-1-naphtyl)-propylamine of the Formula (II) at the desired detection limit can be used. Such a detection method can be selected from the ultraviolet absorption spectrometric, refractometric, fluorescence, mass spectrometric or electrochemical detection methods by a person skilled in the art.

In the high-performance liquid chromatographic separation, several stationary phases known from the prior art are suitable. The criteria for the selection of the stationary phase is that the phase system developed shall comply with the criteria set forth by the pharmacopoeias regarding the high-performance impurity testing analytical methods.

The high-performance liquid chromatographic assay can be performed at room temperature. Alternatively, it is possible to carry out the assay by setting the temperature of the separation column at a temperature between 10 and 60° C.

The flow rate of the mobile phase is determined by the methods known from the prior art. The volume of the introduced sample is determined in a manner that it should not influence the chromatographic separation while at the same time it shall be suitable to achieve the desired detection limit and comply with the criteria of repeatability and reproducibility regarding analytical procedure known according to the prior art.

The sample preparation comprises homogenizing the duloxetine-containing sample, for which the content is approximately known, a portion of the homogenate is weighed with analytical precision, extracted with a solvent or solvent mixture, filtered and the filtrate diluted as necessary.

The high-performance liquid chromatographic assay is carried out according to international guidelines (e.g. ICH Guideline) and the corresponding criteria of pharmacopoeias.

According to a further aspect of the present invention, there are provided medicinal products containing a salt of duloxetine of the Formula (I) with an organic acid according to the present invention, preferably duloxetine citrate, duloxetine fumarate or duloxetine mandelate in admixture with one or more conventional carrier and optionally other pharmaceutical auxiliary agents, which are essentially devoid of (±)-N-methyl-3-(2-thienyl)-3-(4-hydroxy-1-naphtyl)-propylamine of the Formula (II).

The content of the active ingredient in the medicinal product according to the present invention is generally between 0.1-95 percent by weight, preferably 1-50 percent by weight, the most advantageously between 5 and 30 percent by weight.

The medicinal product according to the present invention can be administered orally (in the form of solid pharmaceutical preparations, e.g. powders, tablets, coated tablets, chewing tablets, capsules, microcapsules, granules, dragee, lozenges; or in the form of liquid pharmaceutical products, such as solutions, suspensions or emulsions), parenterally (e.g. in the form of venous, intramuscular, subcutaneous or intraperitoneal injections or in the form of infusions), transdermally (e.g. as patches), as implants or topically (e.g. in the form of creams, ointments or patches). Solid, semisolid or liquid medicinal products according to the present invention can be prepared by the methods known from the state of the art.

Solid medicinal products suitable for oral administration containing a salt of duloxetine of the Formula (I) with an organic acid according to the present invention as active ingredient, which is essentially free from the impurity of the Formula (II), can contain a carrier or vehicle (e.g. lactose, glucose, starch, calcium phosphate, microcrystalline cellulose etc.), a binder (e.g. gelatine, sorbitol, polyvinylpyrrollidone), a disintegrant (e.g. croscarmellose, sodium carboxymethyl cellulose, crospovidone), tabletting aids (e.g. magnesium-stearate, talc, polyethylene glycol, silicic acid, silicon dioxide) or surfactants (e.g sodium laurylsulphate).

Liquid medicinal preparations suitable for oral administration containing a salt of duloxetine of the Formula (I) with an organic acid according to the present invention as active ingredient, which is essentially free from the impurity of the Formula (II), can be solutions, syrups, suspensions or emulsions and can contain suspending agents (e.g. gelatine, carboxymethyl cellulose), emulsifying agents (e.g. sorbitane monooleate), solvents (e.g. water, oils, glycerol, propylene glycol, ethanol), buffers (e.g. acetate, phosphate, citrate buffer) or conserving agents (e.g. methyl-4-hydroxy-benzoate).

Liquid medicinal products suitable for parenteral use containing a salt of duloxetine of the Formula (I) with an organic acid according to the present invention as active ingredient, which is essentially free from the impurity of the Formula (II) are sterile isotonic solutions, which can contain buffers and conserving agents besides the solvent.

Semisolid medicinal products containing a salt of duloxetine of the Formula (I) with an organic acid according to the present invention as active ingredient, which is essentially free from the impurity of the Formula (II), such as suppositories or ointments, contain the active ingredient homogeneously dispersed in the vehicle of the preparation (e.g. polyethylene glycol, cocoa butter).

Medicinal products containing a salt of duloxetine of the Formula (I) with an organic acid according to the present invention as active ingredient, which is essentially free from the impurity of the Formula (II) can be prepared according to the methods of pharmaceutical technology known from the state of the art. The active ingredient is mixed with solid or liquid pharmaceutical carrier and optionally auxiliary agent and transformed into a pharmaceutical dosage form. Vehicles and auxiliary agents, as well as methods form the preparation of the above-mentioned medicinal product are known from the prior art (Remington's Pharmaceutical Sciences, Edition 18, Mack Publishing Co., Easton, USA, 1990).

Medicinal products containing the salt of duloxetine of the Formula (I) with organic acids essentially free from the impurity compound of the Formula (II) contain the active ingredient in dosage units as unit dose.

A further object of the present invention is the use of the salts of duloxetine of the Formula (I) according to the present invention, essentially free from (±)-N-methyl-3-(2-thienyl)-3-(4-hydroxy-1-naphtyl)-propylamine of the Formula (II) for the preparation of medicaments suitable for the treatment or prevention of depression, stress-induced incontinence, neuropathic pain or fibromyalgia, which comprises mixing the duloxetine salt active ingredient with one or more pharmaceutically acceptable vehicle and auxiliary agent and transformed into a pharmaceutical dosage form. Methods for the preparation of such medicament are known per se from the state of the art.

A further object of the present invention is a process for the treatment or prevention of depression, stress-induced incontinence, neuropathic pain or fibromyalgia, which comprises administering a salt of duloxetine of the Formula (I) according to the present invention, essentially free from (±)-N-methyl-3-(2-thienyl)-3-(4-hydroxy-1-naphtyl)-propylamine of the Formula (II) in a therapeutically effective dose to a patient in need of such treatment.

Further details of the present invention are demonstrated in the following examples without restricting the invention to said examples.

### Example 1

### (+)-N-methyl-3-(1-naphtyloxy)-3-(2-thienyl)-propylamine citrate (1:1)

10.0 g (33.3 mmol) duloxetine free base are dissolved in 100 ml of acetone, 6.4 g (33.3 mmol) of citric acid are added at room temperature and the mixture is boiled until the dissolution of the citric acid. The precipitation of the product begins already during the boiling. The suspension thus obtained is cooled and stirred for two hours at room temperature and two hours at the temperature of 0° C. The product is filtered off and washed with acetone.

Yield, 15.0 g (92.1%) white crystalline solid. Optionally the product can be recrystallized from tenfold amount of methanol.

Elemental analysis [calculated on the basis of the Formula C₂₄H₂₇NO₈S (489,6)]:

| | | | | |
|---|---|---|---|---|
| Calculated | C: 58.88 | H: 5.56 | N: 2.86 | S: 6.55 |
| Measured | C: 58.56 | H: 5.55 | N: 2.76 | S: 6.62 |

Melting point, 139-141° C.

Optical purity (HPLC) 99.7%.

The product contains less than 0.01 percent by weight impurity of the Formula (II).

### Example 2

### (+)-N-methyl-3-(1-naphtyloxy)-3-(2-thienyl)-propylamine fumarate (1:1)

10.0 g (33.3 mmol) duloxetine free base are dissolved in 110 ml of acetone, 3.86 g (33.3 mmol) of fumaric acid are added and the mixture is boiled until the dissolution of the fumaric acid. Upon cooling, the product is separated first as an oil and it is crystallized subsequently. The suspension of the crystals is stirred at room temperature for two hours and two hours at the temperature of 0° C., filtered and washed with acetone.

Yield, 13.1 g (96.3%) white crystalline solid.

Elemental analysis [calculated on the basis of the Formula C₂₂H₂₃NO₅S (413.5)]:

| | | | | |
|---|---|---|---|---|
| Calculated | C: 63.91 | H: 5.61 | N: 3.39 | S: 7.75 |
| Measured | C: 63.65 | H: 5.77 | N: 3.36 | S: 7.89 |

Melting point, 138-142° C.

Optical purity (HPLC): 99.6%.

The amount of the impurity of the Formula (II) in the thus obtained product is less than 0.01 percent by weight as determined by HPLC.

### Example 3

### (+)-N-methyl-3-(1-naphtyloxy)-3-(2-thienyl)-propylamine(−)-mandelate (1:1)

10.0 g (33.3 mmol) of duloxetine free base are dissolved in 100 ml of water-free ethanol and 5.0 g (33.3 mmol) of (−)-mandelic acid are added at room temperature. The precipitation of the product begins after a few minutes of stirring. The suspension is stirred for five hours at room temperature and for three hours at the temperature of 0° C., the product is filtered and washed with ethanol.

Yield, 12.6 g (84.2%) white crystalline solid.

Elemental analysis [calculated on the basis of the Formula C₂₆H₂₇NO₄S (449.6)]:

| | | | | |
|---|---|---|---|---|
| Calculated | C: 69.46 | H: 6.05 | N: 3.12 | S: 7.13 |
| Measured | C: 69.25 | H: 6.20 | N: 3.08 | S: 7.14 |

Melting temperature, 72-74.5° C.

Optical purity (HPLC): 99.5%.

The amount of the impurity of the Formula (II) in the product is less than 0.01 percent by weight as determined by HPLC.

### Example 4

### (+)-N-methyl-3-(1-naphtyloxy)-3-(2-thienyl)-propylamine(−) mandelate (1:1)

10.0 g (33.3 mmol) of racemic N-methyl-3-(1-naphtyloxy)-3-(2-thienyl)-propylamine free base are dissolved in 100 ml of diethylether and 3.0 g (20.0 mmol) (−)-mandelic acid are added at room temperature with stirring. After the dissolution of the mandelic acid, the solution is seeded with (+)-duloxetine (−)-mandelate prepared according to Example 3. After a few minutes of stirring, the precipitation of the product begins. The suspension is stirred at room temperature for 20 hours and at the temperature of 0° C. for 10 hours, the precipitated salt is filtered and washed with diethylether.

Yield, 4.30 g (28.9%) white crystalline solid.

Elemental analysis [calculated on the basis of the Formula C₂₆H₂₇NO₄S (449.6)]:

| | | | | |
|---|---|---|---|---|
| Calculated | C: 69.46 | H: 6.05 | N: 3.12 | S: 7.13 |
| Measured | C: 69.21 | H: 6.02 | N: 3.25 | S: 7.02 |

Melting point, 72-74.5° C.

The optical purity of the crude product is 95.5% (HPLC). The crude product can be recrystallized from fivefold volume of ethanol (yield 83.0%). The optical purity of the recrystallized product is 99.5% (HPLC).

The amount of the impurity of the Formula (II) in the thus obtained product is less than 0.01 percent by weight as determined by HPLC.

### Example 5

### Preparation of (+/−)-N-methyl-3-(1-naphtyloxy)-3-(2-thienyl)-propylamine with racemization

The mother lye obtained in the crystallization process of Example 4 is mixed with 30 ml of 10 percent by weight aqueous sodium hydroxide solution with cooling, the diethylether phase is dried over magnesium sulphate and evaporated to dryness.

Yield, 6.5 g yellow oil containing approx. 70.5 percent by weight (−)-N-methyl-3-(1-naphtyloxy)-3-(2-thienyl)-propylamine free base according to HPLC analysis. The oil is mixed with 30 ml of dimethylsulphoxide and 1.0 g potassium hydroxide and the mixture is heated for two hours at the temperature of 105° C. with stirring. The suspension is poured onto the mixture of 50 g of ice and 50 ml of toluene, the phases are separated, the toluene layer is dried and evaporated to dryness.

Yield, 6.1 g (93.8%) yellow oil, which is duloxetine free base racemate according to HPLC analysis.

### Example 6

### (+)-N-methyl-3-(1-naphtyloxy)-3-(2-thienyl)-propylamine hydrochloride (1:1)

A vessel equipped with high-speed stirrer is charged with 200 ml of 2-propanol, 10.0 g (33.3 mmol) duloxetine free base and under intensive stirring, 50 ml of 2-propanol containing 10% hydrochloric acid are added dropwise. The solution is boiled for a few minutes, decolorized, the product precipitated upon cooling is collected at the temperature of 0° C., washed with ethylacetate and dried.

Yield, 14.2 g (85.2%) white crystalline solid.

Elemental analysis [calculated on the basis of the Formula C₁₈H₁₉NOS HCl (333.9)]:

| | | | | |
|---|---|---|---|---|
| Calculated | C: 64.75 | H: 6.04 | N: 4.20 | S: 9.60 |
| Measured | C: 64.62 | H: 5.98 | N: 4.32 | S: 9.54 |

Enantiomer purity: 99.7%.

The amount of the impurity of (±)-N-methyl-3-(2-thienyl)-3-(4-hydroxy-1-naphtyl)-propylamine of the Formula (II) in the thus obtained product is approximately 0.05 percent by weight as determined by HPLC.

### Example 7

### (±)-N-methyl-3-(2-thienyl)-3-(4-hydroxy-naphtyl)-propylamine hydrogenbromide (1:1)

### Hydrogenbromide Salt of the Compound of the Formula (II)

10.0 g (33.3 mmol) of (±)-N-methyl-3-(1-naphtyloxy)-3-(2-thienyl)-propylamine free base are dissolved in 60 ml of 2-propanol and under stirring at room temperature, 16.8 g (68.5 mmol) acetic acid containing 30 percent by weight hydrogen-bromide are added. The mixture is stirred at the temperature of 40° C. for 90 minutes, evaporated in vacuo, the oily residue is washed with two 20 ml portions of diethylether and the diethylether phase is discarded. The residue is crystallized upon the addition of 15 ml of diethylether and 30 ml of water. The crystalline suspension is stirred for 8 hours at the temperature of 0° C., filtered and washed with diethylether.

Yield, 8.24 g (65.4%) white crystalline solid.

Elemental analysis [based on the Formula C₁₈H₂₀BrNOS (378.3)]:

| | | | | | |
|---|---|---|---|---|---|
| Calculated | C: 57.15 | H: 5.33 | Br: 21.12 | N: 3.70 | S: 8.48 |
| Measured | C: 57.19 | H: 5.35 | Br: 21.05 | N: 3.76 | S: 8.50 |

Melting temperature, 244-245° C.

Purity (HPLC) 99.85%

IR(KBr): 3243, 2964, 2784, 2447, 1596, 1515, 1379, 1332, 1258, 1212, 1145, 1065, 1051 cm⁻¹

HNMR (DMSO, i500): 10.14 (s, 1H), 8.55 (s, 2H), 8.19 (dd, 1H), 8.13 (d, 1H), 7.50 (t, 1H), 7.44 (t, 1H), 7.35 (d, 1H), 7.31 (dd, 1H), 7.06 (d, 1H), 6.94 (dd, 1H), 6.91 (d, 1H), 5.09 (t, 1H), 2.90 (m, 2H), 2.54 (t, 3H), 2.48 (q, 2H) ppm.

CNMR (DMSO, i500): 152.54, 148.85, 132.16, 129.27, 126.85, 126.52, 125.16, 124.63, 124.51, 124.45, 123.39, 122.85, 107.73, 47.26, 37.76, 32.78, 32.59 ppm.

COSY: 8.55-2.90-2.54-2.48-5.09, 8.19-7.44-7.50-8.13, 7.35-6.91, 7.31-6.94-7.06 ppm

NOE (10.14 ppm): 8.19, 6.91, 3.40; NOE (8.19 ppm): 10.14, 7.50, 7.44, 3.40 ppm

### Example 8

### (±)-N-methyl-3-(2-thienyl)-3-(4-hydroxy-1-naphtyl)-propylamine free base [free base of the compound of the Formula (II)]

7.82 g (20.6 mmol) of (±)-N-methyl-3-(2-thienyl)-3-(4-hydroxy-1-naphtyl)-propylamine hydrogenbromide prepared according to Example 7 are suspended in 80 ml of water, 2.6 ml of 25 percent by weight aqueous ammonium hydroxide solution are added and the mixture is stirred at the temperature of 40° C. for 90 minutes. The precipitated white crystals are filtered off at room temperature and washed with n-hexane.

Yield, 5.9 g (96.1%) white crystalline solid.

Elemental analysis [calculated on the basis of the Formula C₁₈H₁₉NOS (297.4)]

| | | | | |
|---|---|---|---|---|
| Calculated | C: 72.69 | H: 6.44 | N: 4.71 | S: 10.78 |
| Measured | C: 72.61 | H: 6.48 | N: 4.69 | S: 10.82 |

Melting point, 190-196° C.

IR(KBr): 3447, 3267, 2942, 2441, 1582, 1451, 1385, 1276, 1150 cm⁻¹.

HNMR (DMSO, i500): 8.18 (dd, 1H, J=8.2, 1.1 Hz), 8.13 (d, 1H, J=8.4 Hz), 7.46 (t, 1H, J=7.6 Hz), 7.42 (t, 1H, J=7.4 Hz), 7.31 (d, 1H, J=7.9 Hz), 7.24 (dd, 1H, J=5.1, 1.2 Hz), 6.95 (d, 1H, J=3.5 Hz), 6.89 (dd, 1H, J=5.1, 3.5 Hz), 6.86 (d, 1H, J=7.9 Hz), 5.05 (t, 1H), 2.45 (t, 2H), 2.25 (q, 2H), 2.23 (s, 3H) ppm.

### Example 9

### Assay of (±)-N-methyl-3-(2-thienyl)-3-(4-hydroxy-1-naphtyl)-propylamine of the Formula (II) in duloxetine-containing medicinal products

The analysis is carried out using ultraviolet absorption detector under the following chromatographic conditions:

Column, Chiral OD-R, 250*4,6 mm, 10 μm particle size
Flow rate, 1.0 ml/minute
Injection volume, 10 μl
Column temperature, 40° C.
Temperature of sample compartment, 20° C.
Detection wavelength, 220 nm
Mobile phase A: solution of 40 g of sodium hydroxide and 85 ml of concentrated perchloric acid are dissolved in 200 ml of distilled water, pH 2.1
B: acetonitrile
Eluent composition, isocratic, 55 percent by volume mobile phase A
Analysis time, 30 minutes
Sample concentration, 10 mg/10 ml
Sample solvent, water-acetonitrile 80:20 volume/volume
Needle wash, water-acetonitrile 80:320 volume/volume
Selectivity factor, >4
Number of theoretical plates, min. 10000
Asymmetry factor, <2

The assay is carried out according to the requirements of the pharmacopoeia regarding the determination of the impurities.

### Example 10

### Sample Preparation for the Testing of a Duloxetine-Containing Medicinal Product

The following sample preparation method has been used during the stability testing of duloxetine-containing medicinals products and assay of duloxetine active ingredients.

A sample containing approximately 100 mg of duloxetine is thoroughly homogenized in a mortar, weighed with analytical precision and transferred into a suitable volumetric flask. 80 ml of 80:20 volume by volume mixture of water and acetonitrile are added to the sample and the suspension is shaked in an ultrasonic bath for 10 minutes at the temperature of 25° C. The suspension is filtered and completed to 100 ml with the extraction solvent. A 10-μl portion of the thus obtained solution is analyzed by the high-performance liquid chromatographic method of Example 9.

1-29. (canceled)

30. A salt of [(+)-N-methyl-3-(1-naphthyloxy)-3-(2-thienyl)-propylamine] with an organic acid, which is essentially devoid of the impurity (±)-N-methyl-3-(2-thienyl)-3-(4-hydroxy-1-naphthyl)-propylamine.

31. The salt (+)-N-methyl-3-(1-naphthyloxy)-3-(2-thienyl)-propylamine citrate (1:1), as defined in claim 1, which is essentially devoid of the impurity (±)-N-methyl-3-(2-thienyl)-3-(4-hydroxy-1-naphthyl)-propylamine.

32. The salt (+)-N-methyl-3-(1-naphthyloxy)-3-(2-thienyl)-propylamine fumarate (1:1), as defined in claim 1, which is essentially free from the impurity (±)-N-methyl-3-(2-thienyl)-3-(4-hydroxy-1-naphthyl)-propylamine.

33. The salt (+)-N-methyl-3-(1-naphthyloxy)-3-(2-thienyl)-propylamine(−)-mandelate, as defined in claim 1, which is essentially free from the impurity (±)-N-methyl-3-(2-thienyl)-3-(4-hydroxy-1-naphthyl)-propylamine).

34. N-methyl-3-(2-thienyl)-3-(4-hydroxy-1-naphthyl-propylamine, its isotope-labelled analogs and acid addition salts thereof.

35. A method of assaying a sample of an organic acid salt of [(+)-N-methyl-3-(1-naphthyloxy)-3-(2-thienyl)-propylamine] to determine in the sample an amount of N-methyl-3-(2-thienyl)-3-(4-hydroxy-1-naphthyl)-propylamine, as an impurity in said sample, which comprises the steps of:
(a) chromatographically separating the organic acid salt of [(+)-N-methyl-3-(1-naphthyloxy)-3-(2-thienyl-propylamine] using high pressure liquid chromatography from any impurities contained in the sample;
(b) subjecting the impurities from the sample separated according to step (a) to ultraviolet absorption, spectrometric, refractometric, fluorescent, mass spectrometric or electrochemical analysis to obtain a spectral pattern of the impurities; and
(c) comparing the spectral pattern of the impurities obtained according to step (b) against the spectral pattern of a reference substance selected from the group consisting of N-methyl-3-(2-thienyl)-3-(4-hydroxy-1-naphthyl)-propylamine, its isotope-labeled analogs, and acid addition salts thereof as the reference substance, and determining the presence of N-methyl-3-(2-thienyl)-3-(4-hydroxy-1-naphthyl)-propylamine, based upon a comparison of the spectral patterns from the sample and the spectral pattern from the reference substance.

36. A process for preparing a salt of [(+)-N-methyl-3-(1-naphthyloxy)-3-(2-thienyl)-propylamine] with an organic acid as defined in claims 30, which comprises the step of reacting [(+)-N-methyl-3-(1-naphthyloxy)-3-(2-thienyl)-propylamine] free base in an organic solvent with an organic acid and isolating the thus obtained crystalline salt.

37. The process according to claim 36, wherein citric acid or fumaric acid is used as the organic acid.

38. The process according to claim 36, wherein the salt formation is carried out using a 1.0-1.2 mol-equivalent amount of the organic acid relative to the amount of [(+)-N-methyl-3-(1-naphthyloxy)-3-(2-thienyl)-propylamine] free base.

39. The process according to claim 38, wherein salt formation is carried out in aliphatic esters, ethers or dialkyl-ketones comprising 4 to 10 carbon atoms.

40. The process according to claim 36, wherein (−)-mandelic acid is used as the organic acid.

41. The process according to claim 40, wherein for the preparation of the (−)-mandelate salt of optically pure [(+)-N-methyl-3-(1-naphthyloxy)-3-(2-thienyl)-propylamine], a.0-1.2 molar equivalent amount of the (−)-mandelic acid is used.

42. The process according to claim 40, wherein in the in situ preparation of the optically active (−)-mandelate salt of [(+)-[(+)-N-methyl-3-(1-naphthyloxy)-3-(2-thienyl)-propylamine from racemic N-methyl-3-(1-naphthyloxy)-3-(2-thienyl)-propylamine and (−)-mandelic acid, in the resolution of racemic [(+)-N-methyl-3-(1-naphthyloxy)-3-(2-thienyl)-propylamine, 0.5-1.0 molar equivalent amount of (−)-mandelic acid are used.

43. The process according to claim 36, wherein the salt formation is carried out in aliphatic alcohols comprising 1 to 5 carbon atoms or in aliphatic esters or dialkyl-ketones comprising 4 to 10 carbons atoms.

44. A process for the preparation of N-methyl-3-(2-thienyl)-3-(4-hydroxy-1-naphthyl)-propylamine free base or an acid addition salt thereof, which comprises the step of reacting {(+)-N-methyl-3-(1-naphthyloxy)-3-(2-thienyl)-propylamine] or an acid addition salt thereof in a polar solvent with a strong mineral acid.

45. The process according to claim 44, wherein the N-methyl-3-(2-thienyl)-3-(4-hydroxy-1-naphthyl)-propylamine free base or an acid addition salt thereof is prepared by reacting {(+)-N-methyl-3-(1-naphthyloxy)-3-(2-thienyl)-propylamine] or an acid addition salt thereof with hydrogen bromide.

46. The process according to claim 45 wherein the reaction is carried out in acetic acid, an alcohol comprising 1 to 4 carbon atoms, in water or in a mixture thereof.

47. The process according to claim 45 wherein the reaction is carried out at a temperature between 25 and 100° C.

48. A process for the preparation of {(±)-N-methyl-3-(1-naphthyloxy)-3-(2-thienyl)-propylamine] essentially devoid of N-methyl-3-(2-thienyl)-3-(4-hydroxy-1-naphthyl)-propylamine, as an impurity, which comprises the step of reacting a salt of [(+)-N-methyl-3-(1-naphthyloxy)-3-(2-thienyl)-propylamine] in an organic solvent at elevated temperature with alkali hydroxide.

49. The process according to claims 48 wherein as alkali hydroxide, sodium or potassium hydroxide.

50. The process according to claim 49, wherein either a polar solvent, or a dipolar aprotic solvent is used.

51. The processes according to claim 48 wherein the reaction is carried out at the temperature between 50 to 150° C.

52. A process for the assay of {(+)-N-methyl-3-(1-naphthyloxy)-3-(2-thienyl)-propylamine], its salts and medicinal products containing said compound or its salts, and for indicating stability thereof, which comprises the step of determining the content of (±)-N-methyl-3-(2-thienyl)-3-(4-hydroxy-1-naphthyl-propylamine impurity using a suitable analytical method.

53. The process according to claim 52, wherein (+)-N-methyl-3-(2-thienyl-3-4-hydroxy-1-naphthyl-propylamine impurity content determination is carried out by high-performance liquid chromatography.

54. A pharmaceutical composition comprising a therapeutically effective amount of a salt of [(+)-N-methyl-3-(1-naphthyloxy)-3-(2-thienyl)-propylamine] with an organic acid, which is essentially devoid of the impurity (±)-N-methyl-3-(2-thienyl-3-4-hydroxy-1-naphthyl-propylamine as defined in claim 30 and a pharmaceutically acceptable vehicle or auxiliary agent.

55. A Method for the treatment of depression, stress-induced incontinence, neurophatic pain or fibromyalgia, which comprises the step of administering a therapeutically effective amount of a salt of [(+)-N-methyl-3-(1-naphthyloxy)-2-thienyl-propylamine] with an organic acid, which is essentially devoid of the impurity (±)-N-methyl-3-(2-thienyl)-3-(4-hydroxy-1-naphthyl)-propylamine as defined in claim 30 to a patient in need of such treatment.
